(19) United States
(12) Patent Application Publication
(10) Pub. No.: US 2004/0098096 A1
(43) Pub. Date: May 20, 2004

(54) Endograft device to inhibit endoleak and migration

(75) Inventor: Darwin Eton, Miami, FL (US)

Correspondence Address: NIXON & VANDERHYE, PC, 1100 N GLEBE ROAD, 8TH FLOOR, ARLINGTON, VA, 22201-4714, US

(73) Assignee: The University of Miami, Miami, FL (US)

(21) Appl. No.: 10/680,240

(22) Filed: Oct. 8, 2003

Related U.S. Application Data
(60) Provisional application No. 60/419,974, filed on Oct. 22, 2002.

(51) Int. Cl. A61F 2/06

(52) U.S. Cl. 623/1.13

(57) ABSTRACT

An implantable endograft device which may be characterized as an endograft assembly that is effectively anchored with respect to the weakened blood vessel by filling the aneurysmal sac to preclude further enlargement thereof and to anchor the endograft with respect to the aneurysm. In this way, migration of the endograft is inhibited and exposure of the aneurysmal sac to endoleak circulatory pressures is limited thereby minimizing the risk of vessel wall rupture.

This application claims the benefit of U.S. Provisional Application No. 60/419,974, which was filed Oct. 22, 2002, the disclosure of which is incorporated herein by this reference.

### BACKGROUND OF THE INVENTION

The present invention relates to endograft devices and methods for inhibiting the formation of endoleaks arising from endovascular repair of aneurysms. More particularly, the invention relates to an endograft structure provided as an internal reinforcement for a diseased blood vessel segment and that interfaces with the diseased tissue so as to avoid endoleaks and graft migration.

Blood vessel walls may weaken due to degeneration with aging and atherosclerosis, congenital defect, infection, injury and other conditions. Weakening of a blood vessel wall generally results in a ballooning of the wall referred to as an aneurysm. If left untreated, the aneurysm may rupture and present a life threatening condition for the patient. Aneurysms are the seventh most common cause of death in the United States; 6% of adult men over 70 have aneurysms. Due to the aging of the population, the number of aneurysms is increasing.

Stents are endoprosthetic devices implanted in blood vessels to maintain patency of a constricted region of a blood vessel or to bridge a weakened or aneurysmic region of a blood vessel. Stents that are covered or combined with tubular sleeves are typically referred to as a stent graft or endograft.

Aortic endografts were designed in the 1990's to permit replacement of a diseased vessel segment from within the vessel (endovascularly) in lieu of open surgery. As noted above, an endograft typically includes a graft material and a frame or support structure such as a balloon expandable or self-expanding stent structure. The stent structure may be provided at each end of the graft or may extend along the length of the graft. Endografts are typically introduced percutaneously into the patient's circulatory system on or in a delivery device. More particularly, catheter technology is used to slip a graft into the abdominal aorta. There the endograft either self expands or is balloon expanded to anchor the stent structures at a narrow neck above and below the aneurysm. The graft is held in place by the radial force of the stent against the underlying neck to seal the weakened vessel segment from the circulatory flow. Isolating the aneurysm from the circulatory flow reduces pressure on the weakened vessel wall thereby reducing the likelihood of vascular rupture. Thus, the goal of endograft placement is the complete exclusion of the aneurysmic region from systemic blood flow.

One of the main problems with endovascular grafting is that of continued blood flow into the aneurysm after graft placement, which is referred to in the art as an endoleak. Endoleaks arise either from back bleeding from tributaries into the aneurysmal sac outside the endograft, from blood flow through the, e.g., Dacron polyester sleeve of the graft, or between the prosthesis and the blood vessel after placement of the endograft, e.g. due to improper or incomplete sealing of the graft against the vessel wall and/or due to mechanical failure of the endograft structure. If fluid leaks into the aneurysmal sac, pressure is increased which may result in aneurysmal rupture.

A 20% re-operative rate at 3 years has been reported due to the development of leaks from the native side branches of the artery being excluded intraluminally and due to migration of the device downward.

Another potential complication following endograft implantation, is endograft migration. If the implanted endograft migrates axially of the blood vessel from its position bridging the damaged vessel wall, the damaged vessel wall will be exposed to pressures from the circulating flow increasing the risk of rupture.

### SUMMARY OF THE INVENTION

More particularly, the present invention provides an implantable endograft device which may be characterized as an endograft assembly that is effectively anchored with respect to the weakened blood vessel by filling the aneurysmal sac to preclude further enlargement thereof and to anchor the endograft with respect to the aneurysm. In this way, migration of the endograft is inhibited and exposure of the aneurysmal sac to endoleak circulatory pressures is limited thereby minimizing the risk of vessel wall rupture.

The invention may be embodied in an endovascular device for bridging an aneurysmic region of a blood vessel; comprising: a first, outer graft wall having proximal and distal ends, said outer graft wall being formed from a flexible, elastic material that is selectively expandable to generally conform to an interior shape of the aneurysmic region of the blood vessel; a second, inner graft wall having proximal and distal ends; and at least one stent structure secured to at least one of said inner and outer walls for supporting and securing said respective wall with respect to the blood vessel.

The invention may also be embodied in a method of repairing an aneurysmic region of a blood vessel with an endovascular device, comprising: providing a first, outer graft wall structure; providing a second, inner graft wall structure, said inner and outer graft wall structures defining the endovascular device, said outer graft wall structure being formed from a flexible, elastic material that is selectively expandable to generally conform to an interior shape of the aneurysmic region of the blood vessel; delivering said outer wall structure to the site of said aneurysmic region and securing proximal and distal ends of said outer wall structure with respect to proximal and distal end of said aneurysmic region; delivering said inner wall structure to the site of said aneurysmic region and securing proximal and distal ends of said inner wall structure with respect to proximal and distal ends of said aneurysmic region; filing a space between said inner and outer wall structures by at least one of flowing a solidifiable material to and capturing a solidifiable material in said space; and allowing said material to solidify thereby to anchor the endovascular device defined by said inner and outer graft wall structures with respect to said aneurysmic region.

### BRIEF DESCRIPTION OF THE DRAWINGS

These and other objects and advantages of this invention will be more completely understood and appreciated by careful study of the following more detailed description of the presently preferred exemplary embodiments of the invention, taken in conjunction with the accompanying drawings, in which:

FIG. 1 is a schematic cross sectional view of a first embodiment of an endograft assembly according to the invention disposed in an aneurysmic blood vessel;

FIG. 2 is a schematic cross sectional view of the endograft assembly of FIG. 1, showing expansion of the graft outer wall or membrane;

FIG. 3 is a schematic cross sectional view of the endograft assembly of FIG. 1, with the graft outer membrane fully expanded to fill the aneurysmic sac;

FIG. 4 is a schematic cross sectional view of a second embodiment of an endograft assembly according to the invention disposed in an aneurysmic blood vessel;

FIG. 5 is a schematic cross sectional view of the endograft assembly of FIG. 4, showing expansion of the graft outer membrane;

FIG. 6 is a schematic cross sectional view showing the placement of a first, outer graft wall or membrane as a first step in the placement of an endograft assembly according to a third embodiment of the invention;

FIG. 7 is a schematic cross sectional view of the graft structure of FIG. 6, expanded to line the aneurysmic sac;

FIG. 8 is a schematic cross sectional view showing the placement of a second, inner graft wall or membrane as a final step in the placement of an endograft assembly according to the third embodiment; and

FIG. 9 is a schematic cross sectional view of a bifurcated endograft assembly embodying the invention.

### DETAILED DESCRIPTION OF THE INVENTION

The present invention relates to endograft devices and methods for inhibiting migration of endografts and/or the formation of endoleaks arising from endovascular repair of aneurysms.

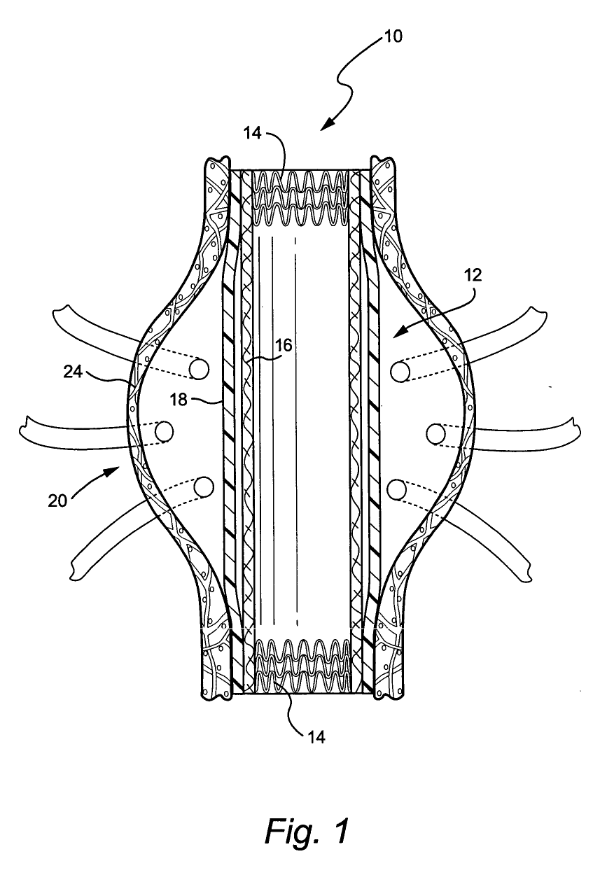

With reference to the embodiment illustrated in FIG. 1, the endograft device or assembly 10 provided in accordance with the invention may be generically characterized as comprised of a generally tubular graft main body 12 having a support frame or stent 14 for engaging the healthy blood vessel tissue on upstream and downstream ends of the aneurysm and supporting and anchoring the endograft with respect thereto. The stent structure may extend the entire length of the endograft structure or may be provided by generally discrete stent bands at proximal and distal ends of the endograft assembly, as in the illustrated embodiment. The stent may be located external or internal to the graft material, or within the graft material itself. The stent structure is preferably and advantageously formed from nitenol or another known temperature responsive, self-expanding memory metal. In the alternative, the stent structure may be provided as an expandable mesh, or other expandable configuration, that is adapted to be mechanically expanded at the target site in the blood vessel by, e.g., the inflatable balloon of a conventional delivery catheter. As a variety of stent/endograft delivery catheters are known in the art and may be adapted to deliver the endograft of the invention, and for clarity, the delivery catheter is not illustrated in the accompanying drawings.

The endograft main body is comprised of inner and outer structural walls or membranes 16,18. The inner wall or membrane is provided to define a flow passage for circulating blood. As such, the inner wall 16 is formed as a tube of predetermined deployed diameter. The outer wall or membrane 18 is comprised of generally tubular, flexible material that is adapted to expand to generally conform to the interior surface of the aneurysm sac 20 so to define with the inner membrane layer a fillable space 22 within the endograft assembly. When the fillable space is filled with blood, saline or a polymeric material, the bulbous configuration of the endograft assembly precludes migration of the endograft and inhibits endoleaks from undesirably exposing the aneurysmic wall 24 to pressures that may lead to rupture. Thus, the inner cylindrical surface of the graft delimits the blood lumen whereas the outer wall 18 of the graft expands to generally fit the topography of the aneursymic vessel wall 24. The space 22 between the membranes may either be filled with blood or with an externally administered fluid. The intent is to fill and seal off the aneursymal space safely without embolizing distally into important side branches. Moreover, the graft will be effectively seated, inhibiting buckling and slippage.

In accordance with a first adaptation of the invention, the endograft assembly is comprised of a double-walled main body, with the ends integrated to define a one piece endograft structure.

FIGS. 1-3 illustrate a first embodiment of an endograft device 10 that is provided in accordance with the first adaptation of the invention, secured with respect to the aneursymic vessel. According to this embodiment, the inner wall or membrane 16 of the endograft is formed from an material that has small interstices between fibers or has pores to allow blood to gradually flow or leak out into the space 22 between the layers of the graft structure, as described more particularly below. An example of a suitable material for the porous inner wall of the endograft assembly is knitted Dacron. The inner wall structure is sized and configured to define a circulatory flow passage of prescribed diameter, generally corresponding to the diameter of the upstream and downstream healthy segments of the blood vessel.

As noted above, the endograft is further comprised of a second, outer wall or membrane 18. The outer membrane of the endograft structure is defined by an expandable, impermeable material, such as unsupported polytetrafluroethyene (goretex) or polyurethane carbonate which when unstressed collapses so as to be disposed in close proximity to the inner wall, but which may be expanded to define a receptacle or fillable space 22 with the inner layer.

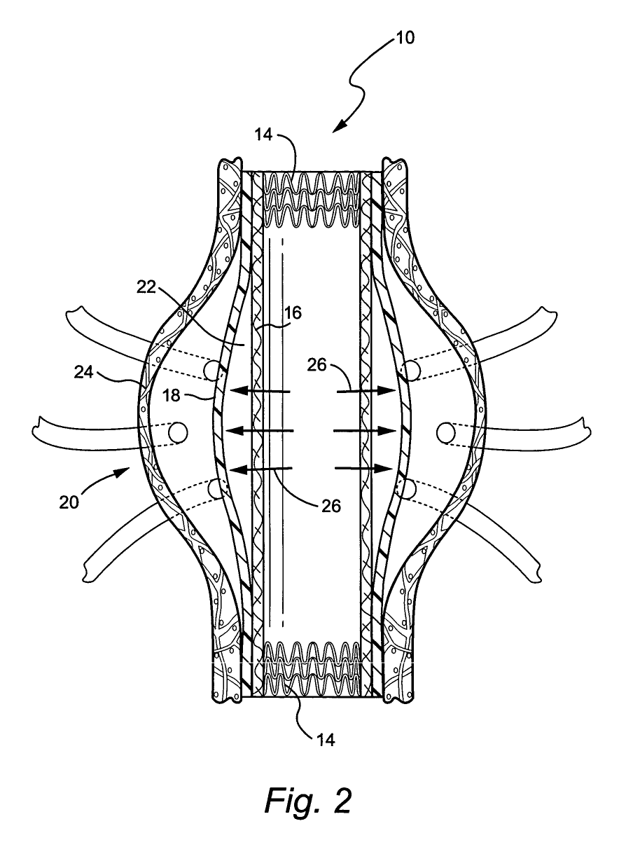

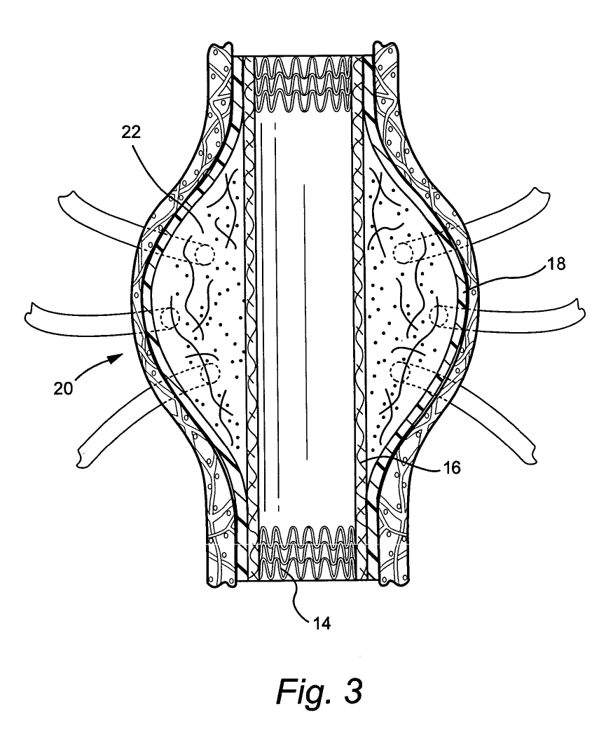

Once the endograft has been placed in a target portion of the blood vessel to bridge a weakened wall portion that has ballooned, since the inner wall is temporarily permeable and not able to expand away from the stent, blood 26 initially permeates the porous inner wall 16 material to enter the space 22 between the expandable outer wall 18 and the porous inner wall 16. The space between the inner and outer walls becomes larger (FIG. 2) as blood under pressure from the circulatory system flows thereinto until the expandable material engages the (weakened) blood vessel wall 24 (FIG. 3). As is apparent, the patient's own blood pressure does the work of expansion of the outer membrane. The outer material is advantageously soft, relatively impervious, and elastic. Moreover, the outer membrane is preferably capable of expanding to accommodate the aneurysmic space, irrespective of volume. In this regard, a pre-defined or limited volume would be less desirable because the aneurysmic space will typically have an irregular topography and it would be particularly advantageous for the outer membrane to fit the topography closely.

The permeability of the inner membrane is temporary by virtue of the fact that after blood flows through the material, the blood will clot within it, filling the interstices, and ultimately make the wall impervious. Further expansion of the outer membrane stops as soon as the aneurysmic space is filled and/or as soon as clot formation occurs. This happens after the heparin needed for the procedure to prevent clot formation is consumed (half life is 90 minutes) or reversed chemically with protamine. In addition, as the inner material interstices seal, there is no longer any flow through it into the space 22 between it and the outer membrane.

As noted above, once the space defined between inner and outer layers has become filled with blood and the outer layer of expansible material conforms to the shape of the aneurysm, the blood in the space eventually clots and the pores of the inner layer of the endograft are sealed with thrombus. The clot thus stabilizes the graft position to prevent migration and fills the space otherwise vulnerable to endoleakage without undesirably stressing the weakened vessel wall.

The issue of endoleak at the ends of conventional endografts is commonly treated by graft modular extension, i.e., placing another device. However, if the aneurysmic space is completely filled in accordance with the invention, any endoleak at the proximal and/or distal ends would have no where to go and should clot in the area of the endoleak.

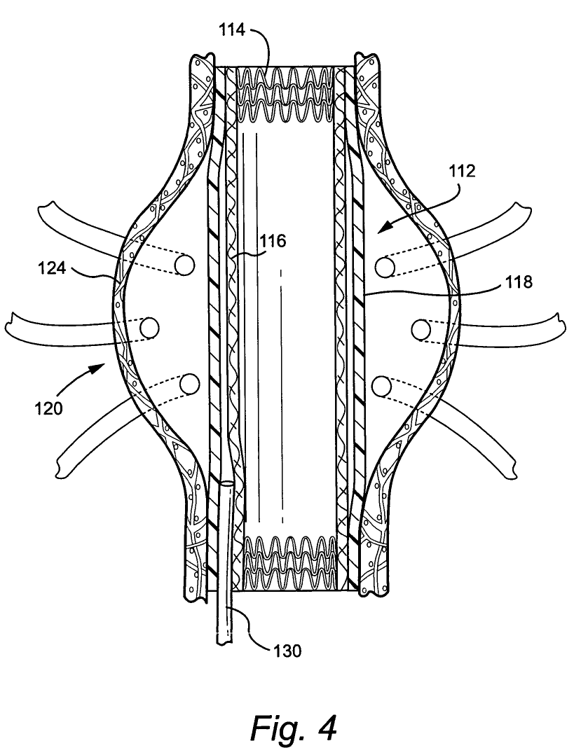

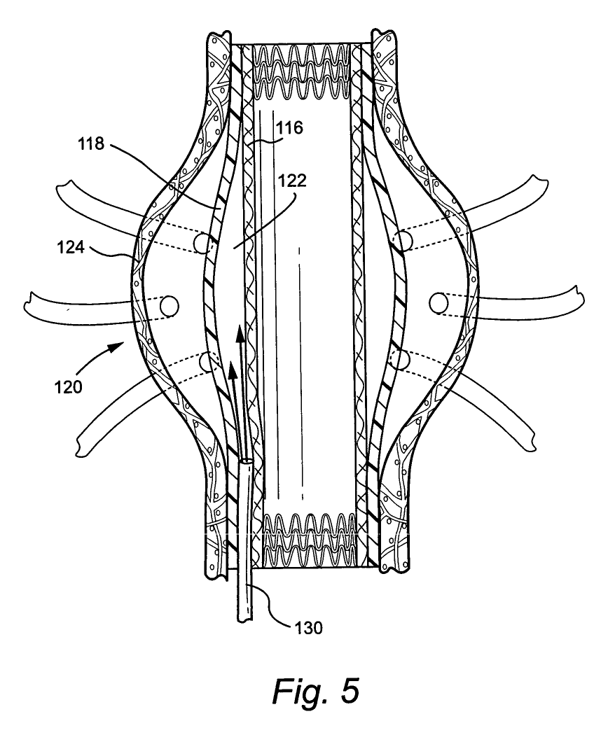

A second alternative embodiment of the first adaptation of the invention is illustrated by way of example, in FIGS. 4-5. The endograft structure of this embodiment is a double wall structure generally similar to the embodiment of FIG. 1. Accordingly, corresponding structures are identified with corresponding reference numbers, incremented by 100, but are not discussed in detail except as appropriate to call out the characteristics of the second embodiment.

In this embodiment, the space 122 between the inner and outer wall or membranes 116, 118 is fillable via a small catheter 130. In one example, a port is defined between the outer and inner layers of the endograft adjacent one axial end of the fillable intraluminal space and a small catheter, separate from the delivery catheter, is disposed in communication with the port. Once the endograft has been placed, a suitable biocompatible fluid can then be injected from the outside through the catheter 130 into the intraluminal space 122 to appose the outer membrane 118 against the inner aneurysm surface, and then solidify. The space may, for example, be filled with blood which would then clot. Another alternative is to fill the space with plasma and cryoprecipitate and then infuse calcium and thrombin to make a firm glue, e.g., BIOglue. As a further alternative, there are liquids used in neurointerventional radiology, that immediately solidify at body temperature, that could be adapted to use in the invention. In this embodiment, the blood flow lumen defining inner wall or membrane of the endograft may be (temporarily) porous or non-porous.

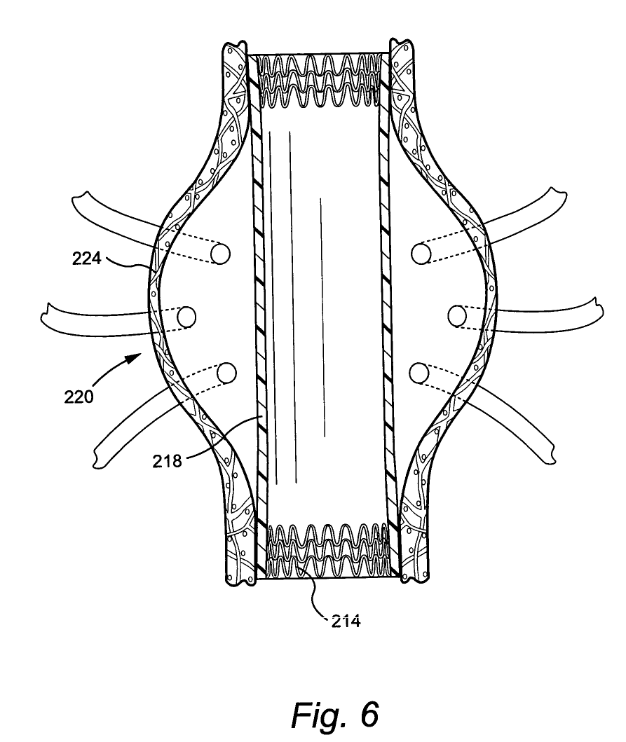

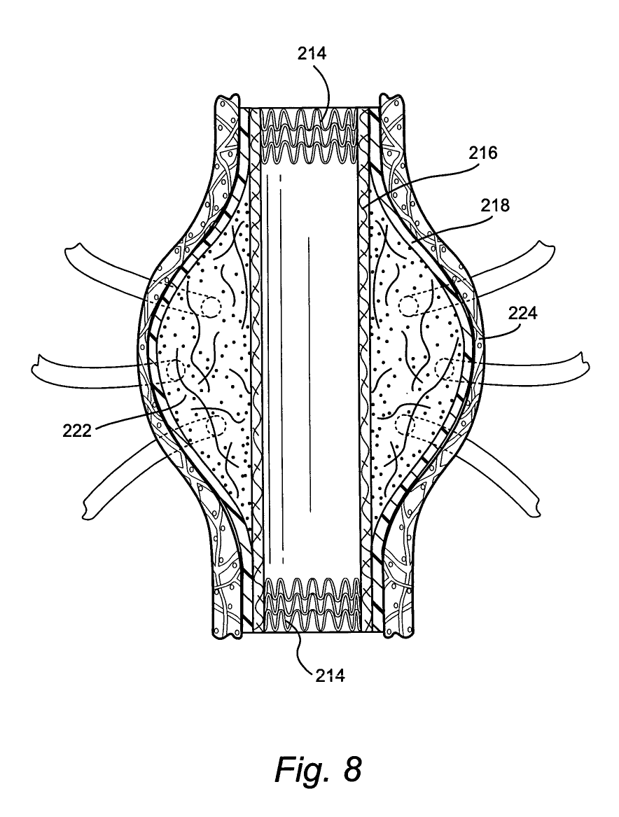

A second adaptation and third embodiment of the invention is illustrated in FIGS. 6-8. As can be seen, this embodiment is similar to the embodiments of FIGS. 1-5 except that rather than providing the endograft assembly as an integrated structure, two entirely separate endografts 216, 218 are placed concentrically, one to line the aneurysmic sac 220 and the second to define the passage for circulatory flow. Again reference numbers corresponding to those used in the first embodiment are used but incremented by 200.

In this embodiment, the first placed, outer endograft 218 is generally tubular having proximal and distal ends and a flexible membrane extending therebetween. The proximal and distal ends are stent supported as at 234 to anchor the endograft to the healthy tissue upstream and downstream of the aneurysmic vessel wall 224. The flexible membrane intermediate the proximal and distal ends is a readily expansible graft material that can conform to the inner circumferential surface of the aneurysmal sac, such as a material as described above for the second, outer wall or membrane of the endograft assembly of the first and second embodiments.

The second placed, inner stent supported endograft 216 is implanted concentrically to the first endograft 218, but defines a generally constant inner cross-sectional passage for circulatory flow. The second endograft may be stent 214 supported at proximal and distal ends or the stent structure thereof may extend along substantially the entire length thereof. Once placed, second endograft also defines an intraluminal space 222 with the first endograft.

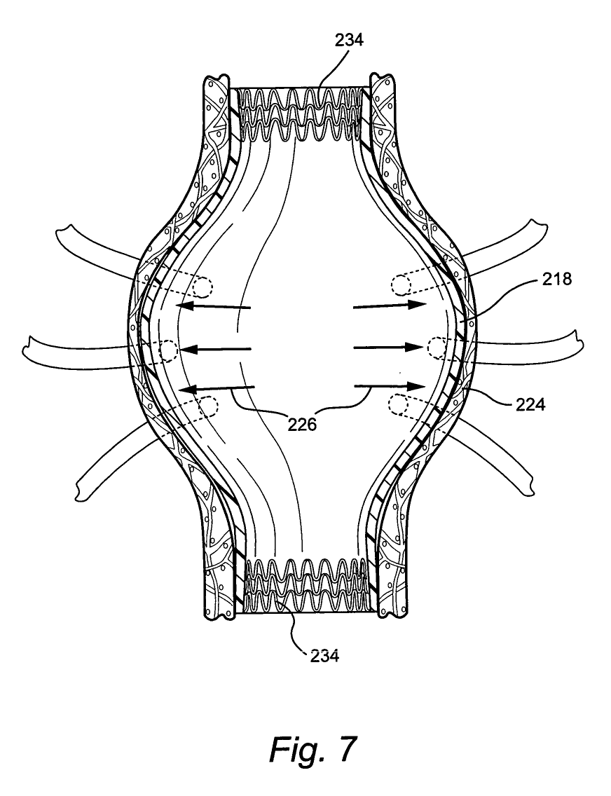

In this embodiment, the first, outer endograft structure is readily expansible when exposed to the patient's blood pressure (FIG. 7). Thus, apart from the short stent supported portions 234 at proximal and distal ends, provided for anchoring purposes (balloon expandable or self expandable), the body of the graft is totally elastic and will balloon out to meet the inner surface of the aneurysmic wall 224. Accordingly, after deployment of the first, outer endograft structure, as illustrated in FIG. 6, the graft can and will expand out due to the patient's own blood pressure (as shown by arrows 226) to meet the inner surface of the aneurysm, as shown in FIG. 7. The space defined within the outer wall will be filled with blood at this point.

A second endograft 216, which may be of conventional design, is then placed concentrically to the first endograft, thus capturing blood in the pocket or space 222 between the two grafts. The captured blood clots in due course, as in the first embodiment, so that the aneurysmic sac 220 is filled and migration of the endograft is prevented.

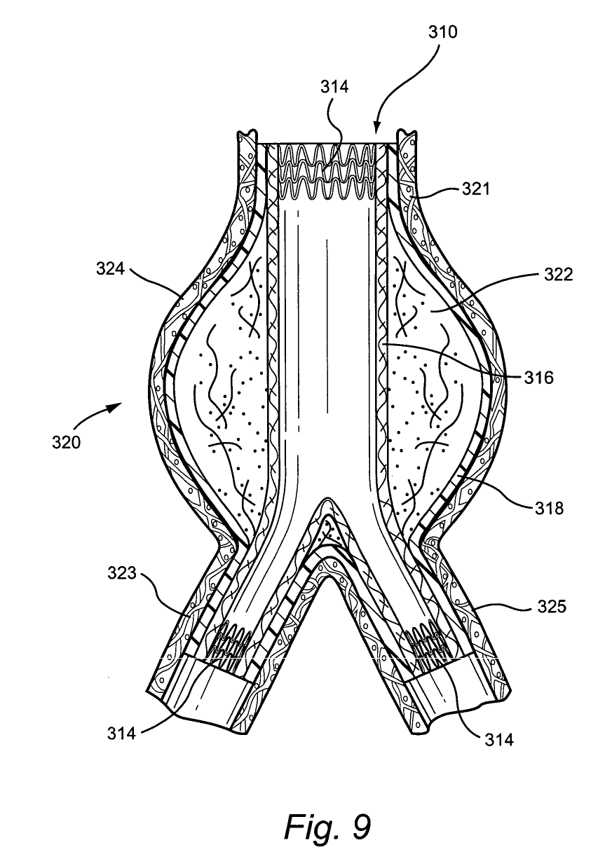

It should be noted that while a single lumen tubular endograft is illustrated in FIGS. 1-8 and has been described above, the invention may also be adapted as a bifurcated graft. More particularly, aneurysms often form in the abdominal aorta immediately proximal to the common iliac arteries. FIG. 9 schematically illustrates an aortic aneurysm in this region. Here the aorta 321 can be seen branching distally into the right iliac artery 323 and the left iliac artery 325. Proximal from the iliac arteries 323, 325, an aortic aneurysm 320 can be seen as a bulging section of the aorta 321. Although not depicted in FIG. 9, such an aneurysm may even extend down one or both iliac arteries. In this embodiment, the endograft 310 is generally shaped as an inverted Y, with a stent 314 provided at least at proximal and distal ends of the assembly. As in the above-described embodiments, the stent structure 314 may extend the entire length of the endograft structure or may be provided by generally discrete stent bands at each of the proximal and distal ends of the endograft assembly, as in the illustrated embodiment, and may be self-expanding or balloon expandable.

The main body of the endograft 310 of the FIG. 9 embodiment is comprised of inner and outer walls or membranes 316, 318 that define a fillable space 322 therebetween. In this respect, the endograft 310 of the FIG. 9 embodiment generally corresponds to the embodiments described above with reference to FIGS. 1-8 and can be defined, implanted, and filled to anchor the endograft with respect to the wall 324 of the aneurysm in a like manner.

While the invention has been described above with reference primarily to the treatment of aneurysms within the chest or abdomen, it is to be understood that a miniaturized version of the invention could be used in other portions of the circulatory flow, including possibly in the brain.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. An endovascular device for bridging an aneurysmic region of a blood vessel; comprising:
a first, outer graft wall having proximal and distal ends, said outer graft wall being formed from a flexible, elastic material that is selectively expandable to generally conform to an interior shape of the aneurysmic region of the blood vessel;
a second, inner graft wall having proximal and distal ends; and
at least one stent structure secured to at least one of said inner and outer walls for supporting and securing said respective wall with respect to the blood vessel.

2. An endovascular device as in claim 1, wherein first and second stent structures are provided adjacent proximal and distal ends of said respective wall.

3. An endovascular device as in claim 1, wherein first and second stent structures are secured to each of said inner and outer walls adjacent said proximal and distal ends thereof.

4. An endovascular device as in claim 1, wherein each of said inner and outer walls is substantially free from said stent structure intermediate proximal and distal end portions thereof.

5. An endovascular device as in claim 1, wherein said inner wall is formed of a material that is at least one of permeable to blood or has pores defined therethrough for the passage of blood.

6. An endovascular device as in claim 1, further comprising a catheter in communication with a space between said inner and outer graft walls and sealed with respect to said inner and outer graft walls for selectively filling said space.

7. An endovascular device as in claim 1, wherein said material of said outer wall structure is one of polytetrafluroethyene and polyurethane carbonate.

8. An endovascular device as in claim 1, wherein a material of said inner graft structure is knitted Dacron polyester.

9. An endovascular device as in claim 1, wherein said stent structure is a self-expanding stent structure.

10. A method of repairing an aneurysmic region of a blood vessel with an endovascular device, comprising:
providing a first, outer graft wall structure;
providing a second, inner graft wall structure, said inner and outer graft wall structures defining the endovascular device, said outer graft wall structure being formed from a flexible, elastic material that is selectively expandable to generally conform to an interior shape of the aneurysmic region of the blood vessel;
delivering said outer wall structure to the site of said aneurysmic region and securing proximal and distal ends of said outer wall structure with respect to proximal and distal ends of said aneurysmic region;
delivering said inner wall structure to the site of said aneurysmic region and securing proximal and distal ends of said inner wall structure with respect to proximal and distal ends of said aneurysmic region;
filing a space between said inner and outer wall structures by at least one of flowing a solidifiable material to and capturing a solidifiable material in said space; and
allowing said material to solidify thereby to anchor the endovascular device defined by said inner and outer graft wall structures with respect to said aneurysmic region.

11. A method as in claim 10, wherein said steps of delivering said outer graft structure and delivering said inner graft structure are performed simultaneously with said inner wall structures disposed concentrically within the outer wall structure.

12. A method as in claim 10, wherein said steps of delivering said outer graft structure and delivering said inner graft structure are performed sequentially.

13. A method as in claim 10, wherein said step of filling said space comprises providing an inner graft structure that is at least one of permeable to blood or has pores defined therethrough for the passage of blood and allowing blood to flow through said inner graft structure into said space.

14. A method as in claim 10, wherein said step of filling said space comprises providing a catheter in communication with the space between said inner and outer wall structures and sealed with respect to said inner and outer wall structures and delivering said solidifiable material through said catheter to said space.

15. A method as in claim 10, wherein said solidifiable material is blood.
